(19) United States
(12) Patent Application Publication
(10) Pub. No.: US 2010/0173344 A1
(43) Pub. Date: Jul. 8, 2010

(54) METHODS FOR ISOLATING AND USING PITUITARY ADENOMA STEM CELLS AND PITUITARY ADENOMA CELLS

(75) Inventor: John S. Yu, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/438,123

(22) PCT Filed: Aug. 22, 2007
(86) PCT No.: PCT/US07/76505

§ 371 Date: Feb. 19, 2009

Related U.S. Application Data
(60) Provisional application No. 60/823,433, filed on Aug. 24, 2006.

(51) Int. Cl. C12Q 1/02; C12N 5/00; C12N 5/02; C12N 15/09

(52) U.S. Cl. 435/29; 435/325; 435/378; 435/377; 435/69.4

(57) ABSTRACT

The present invention describes pituitary adenoma stem cells, pituitary carcinoma stem cells, a method of obtaining the stem cells, and a method of using the stem cells. Uses of the pituitary stem cells include but are not limited to producing pituitary hormones and identifying drugs to treat pituitary disease conditions or pituitary-related disease conditions.

### FIELD OF INVENTION

This invention relates to methods of isolating and using pituitary adenoma stem cells.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Pituitary adenomas are typically slow growing, benign neoplasm of epithelial origin. They account for about 10% of intracranial neoplasms, and are present during early adulthood. They often remain undiagnosed and are found in 6-24% of adult autopsies. The clinical presentation of pituitary adenomas varies depending on the location and severity of the tumor. The classification of these tumors is based on plasma hormone levels or immunohistochemical staining. Prolactinomas are the most common; they cause amenorrhea, galactorrhea, infertility in females and hypogonadism in males. Somatotrophic adenomas secrete an excess of growth hormone and cause gigantism in children and acromegaly in adults. Adrenocorticotropic hormone (ACTH)-secreting adenomas produce Cushing's disease. Gonadrotophic (secreting luteinizing hormone and follicle-stimulating hormone) and thyrotropic adenomas are rare and the latter cause hyperthyroidism. Some diagnosed pituitary adenomas do not secrete hormones and they are classified as null cell adenomas and the diagnosis is made on the basis of visual difficulties arising from the compression of the optic nerve. The classification of pituitary adenomas depends also on the size of the tumors: macroadenomas are greater than 10 mm in diameter and microadenomas have a diameter less than 10 mm.

A stem cell is a cell type that has a unique capacity to renew itself and give rise to specialized or differentiated cells. Although most cells of the body are committed to conduct a specific function, a stem cell is uncommitted until it receives a signal to develop into a specialized cell type. What makes the stem cells unique is their proliferative capacity, combined with their ability to become specialized. Somatic stem cells are present in the adult organism. Pluripotency tests have shown that whereas the embryonic or blastocyst-derived stem cells can give rise to all cells in the organism, including the germ cells, somatic stem cells have a more limited repertoire in descendent cell types.

Isolation of pituitary stem cells from a normal pituitary or adenoma, and cell lines from a pituitary adenoma have never been described. A human pituitary-derived folliculostellate cell (“FSC”) line was developed spontaneously from the culture of a clinically nonfunctioning pituitary gonadotroph adenoma. (Danila et al., A Human Pituitary Tumor-derived Folliculostellate Cell Line, J. Clin. Endocrin. & Metab. (2000), 85(3), pp. 1180-1187.) Danila et al. hypothesized that the transformation and immortalization of the line were possibly due to mutation of the p53 gene. This represented the first time that a pituitary-derived FSC line was developed. However, the cell line was not a pituitary-derived stem cell line developed by a reliable method from a pituitary tumor. In fact, the cells described by Danila at al. do not have any characteristics of stem cells. Danila at al. never asserted that their cell line was based on stem cells, and affirmatively characterized them as FSCs with the attendant characteristics of those cells.

The isolation and identification of putative adult stem cells of the normal rat pituitary have been described in the art. (Chen et al., The Adult Pituitary Contains a Cell Population Displaying Stem/Progenitor Cell and Early-embryonic Characteristics, Endocrin. (2005), 146(9), pp. 3985-3998.) Chen et al. identified a population of cells that clonally replicated to non-adherent spheres and expressed candidate stem cell markers, signaling molecules known to be involved in stem cell renewal and fate decision, and pituitary-related embryonic signals and transcription factors. Chen et al. noted that these characteristics are suggestive of an early embryonic and stem/progenitor cell phenotype. However, they also offered alternative suggestions to explain the stem cell characteristics, i.e., that the cells had dedifferentiated and were capable of generating new hormone-producing cells using a program mimicking the embryonic process, or that the cells were intermediates in transdifferentiation events that may have occurred via an immature (uncommitted) state. Also, in Krylyshkina et al., (Nestin-immunoreactive Cells in Rat Pituitary Are neither Hormonal nor Typical Folliculo-stellate Cells, Endocrin. (2005), 146(5), pp. 2376-2387) the authors identified nestin immunoreactivity in scattered cells of the anterior, intermediate, and neural lobes of the rat pituitary. The cells were not hormonal or typical FSCs; indeed, the authors in Krylyshkina et al. were unable to identify the cells. Nestin is a marker for stem/progenitor cells, but is also present in many other cell types.

Reviews of the morphological characteristics of FSCs have been published. (Horvath et al., Folliculo-stellate Cells of the Human Pituitary: A Type of Adult Stem cell?, Ultrastructural Path. (2002), 26, pp. 219-228; Inoue et al., The Structure and Function of Folliculo-Stellate Cells in the Anterior Pituitary Gland, Arch. Histol. Cytol. (1999), 62(3), pp. 205-218.) These reviews suggest that FSCs may be “a kind of stem cell” with the potential to differentiate into endocrine cells, or which are involved in this differentiation. However, Horvath et al. noted that one problem with this assertion was the lack of evidence of the multiplication of FSCs. Mitoses are very rare in the pituitary, even with hyperplasia. During the 30 years of experience in pituitary research, Horvath et al. had not discovered a mitotic FSC, and found only one article reporting cell division in the FSC type. Inoue et al. also noted a problem with the assertion that FSCs may be a kind of stem cell, which is that it had been reported that FSCs were not a homogeneous cell type, but were heterogeneous. Thus, Inoue et al. could not discount the possibility that FSCs constituted a group of cells that were both functionally and ontogenically heterogeneous.

Notwithstanding the above, Chen et al. described these putative stem cells in rats and not in humans, and they did not identify or develop an adenoma stem cell line isolated from a tumor. The research was not conducted on human pituitary cells, and an adenoma stem cell line was not developed from a pituitary tumor. Furthermore, neither Horvath et al. nor Inoue et al. disclosed the identification, isolation, or preparation of a pituitary adenoma stem cell or stem cell line.

Thus, there exists a need in the art for a method of isolating and/or generating pituitary adenoma stem cells and cell lines; pituitary adenoma cells and cell lines; pituitary carcinoma stem cells and cell lines; and pituitary carcinoma cells and cell lines. These cells and cell lines will be useful to test therapeutic products in vitro; study pituitary adenomas, pituitary carcinomas, pituitary conditions and disease conditions, and pituitary-related conditions and disease conditions; and obtain human pituitary hormone products (e.g., prolactin, human growth hormone, adrenocorticotropic hormone, and sexual hormones such as luteinizing hormone and follicle-stimulating hormone).

### SUMMARY OF THE INVENTION

The following embodiments and aspects thereof are described and illustrated in conjunction with compositions and methods which are meant to be exemplary and illustrative, not limiting in scope.

The present invention provides for an isolated pituitary stem cell comprising cell markers selected from nestin, CD133 or both. In one embodiment, the pituitary stem cell may be a pituitary adenoma stem cell. The pituitary stem cell may be from a pituitary adenoma selected from prolactinoma, somatotrophic adenoma, adrenocorticotropic hormone (“ACTH”)-secreting adenoma, gonadrotophic adenoma, thyrotropic adenoma, null cell adenoma, or combinations thereof. In another embodiment, the pituitary stem cell may be a pituitary carcinoma stem cell.

In various embodiments, the isolated pituitary stem cell may be obtained by providing pituitary tumor tissue; washing the pituitary tumor tissue; dissecting the pituitary tumor tissue; digesting the pituitary tumor tissue; triturating the pituitary tumor tissue to dissociate pituitary cells; culturing the pituitary cells in a medium comprising EGF and bFGF; and selecting the pituitary cells growing as spheres.

The invention also provides for a method of obtaining a population of pituitary cells comprising providing a population of pituitary stem cells and culturing the population of pituitary stem cells in a differentiation culture medium wherein the population of pituitary stem cells are induced to differentiate into pituitary cells. The differentiation culture medium may comprise DMEM/F12, glutamine, horse serum, and fetal bovine serum. The pituitary cells used in the method may be pituitary adenoma cells. In various embodiments, the pituitary adenoma cells used in the method may be selected from prolactinoma, somatotrophic adenoma, adrenocorticotropic hormone (“ACTH”)-secreting adenoma, gonadrotophic adenoma, thyrotropic adenoma, null cell adenoma, or combinations thereof. In another embodiment, the pituitary cells used in the method may be pituitary carcinoma cells.

The present invention further provides for a method of producing a pituitary hormone, comprising providing a population of pituitary stem cells and/or pituitary cells obtained by differentiation of pituitary stem cells; culturing the population in a culture medium; and isolating the pituitary hormone from the culture medium or the intracellular contents of the pituitary stem cells and/or pituitary cells obtained by differentiation of pituitary stem cells. The pituitary hormone produced may be selected from prolactin, growth hormone, adrenocorticotropic hormone, sexual hormone, or combinations thereof. The population of cells used to produced the pituitary hormone may be selected from prolactinoma, somatotrophic adenoma, adrenocorticotropic hormone (“ACTH”)-secreting adenoma, gonadrotophic adenoma, thyrotropic adenoma, null cell adenoma, carcinoma or combinations thereof.

The invention also provides for a method of identifying a drug to treat a pituitary disease condition or a pituitary-related disease condition, comprising: providing a population of pituitary stem cells and/or pituitary cells obtained by differentiation of pituitary stem cells; culturing the population in a culture medium; adding a test compound to the culture medium; and determining the effect of the test compound on the population, wherein a test compound having a desired effect is identified as a drug capable of treating the pituitary disease condition or pituitary-related disease condition. The pituitary disease condition or pituitary-related disease condition may be selected from pituitary adenoma, pituitary carcinoma, amenorrhea, galactorrhea, infertility, hypogonadism, gigantism, acromegaly, Cushing's disease, hyperthyroidism or combinations thereof. The pituitary stem cells may be obtained from a pituitary adenoma or a pituitary carcinoma. In various embodiments, the pituitary adenoma may be selected from prolactinoma, somatotrophic adenoma, adrenocorticotropic hormone (“ACTH”)-secreting adenoma, gonadrotophic adenoma, thyrotropic adenoma, null cell adenoma, or combinations thereof. In various embodiments, the population of pituitary stem cells may be obtained by: providing pituitary tumor tissue; washing the pituitary tumor tissue; dissecting the pituitary tumor tissue; digesting the pituitary tumor tissue; triturating the pituitary tumor tissue to dissociate pituitary cells; culturing the pituitary cells in a medium comprising EGF and bFGF; and selecting the pituitary cells growing as spheres.

In other embodiments, the population is a population of pituitary carcinoma stem cells and/or pituitary carcinoma cells obtained by differentiation of pituitary carcinoma stem cells and the drug is an anti-cancer drug.

The present invention also provides for a kit for producing a pituitary hormone using pituitary adenoma stem cells, comprising: a population of pituitary stem cells and/or pituitary cells obtained by differentiation of pituitary stem cells; instructions to use the population to produce the pituitary hormone comprising: instructions to culture the population in a culture medium; and instructions to isolate the pituitary hormone from the culture medium or the intracellular contents of the population. The pituitary hormone produced by the kit may be prolactin, growth hormone, adrenocorticotropic hormone, sexual hormone or combinations thereof. The population in the kit may be a population of cells selected from prolactinoma, somatotrophic adenoma, adrenocorticotropic hormone (“ACTH”)-secreting adenoma, gonadrotophic adenoma, thyrotropic adenoma, null cell adenoma, carcinoma or combinations thereof.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are considered illustrative rather than restrictive.

FIG. 1 depicts pituitary adenoma cells in accordance with an embodiment of the present invention. (A) Pituitary adenoma stem cells. (B) After withdrawal of growth factors, cells start to differentiate. (C) Daughter cells growing adherent in presence of 10% fetal bovine serum.

FIG. 2 depicts immunocytochemistry of pituitary adenoma cells in accordance with an embodiment of the present invention. Differentiated pituitary adenoma stem cells were subjected to immunostaining with the following antibodies: (A) Nestin; (C) β-III tubulin; (E) GFAP; and (G) S100. B, D, F and H depict the corresponding DAPI staining.

FIG. 3 depicts prolactin concentration in pituitary adenoma stem cells in accordance with an embodiment of the present invention. Tumor stem cells and daughter adherent cells from pituitary adenomas were plated at the density of 5×10⁵ cells/ml. The conditioned medium was collected at different time points as indicated in the graph and subjected to ELISA immunoassay. The results are the average±standard deviation of two independent experiments and all values are calculated by subtracting the blank and the value of un-conditioned medium.

FIG. 4 depicts growth hormone concentration in pituitary adenoma stem cells in accordance with an embodiment of the present invention. Tumor stem cells and daughter adherent cells from pituitary adenomas were plated at the density of 5×10⁵ cells/ml. The conditioned medium was collected at different time points as indicated in the graph and subjected to ELISA immunoassay. The results are the average±standard deviation of two independent experiments and all values are calculated by subtracting the blank and the value of un-conditioned medium.

FIG. 5 depicts semiquantitative RT-PCR in pituitary adenoma stem and daughter adherent cells in accordance with an embodiment of the present invention. RNA extraction and retro-transcription were performed as described herein. As controls, RNA from normal human liver (negative control) and normal human pituitary (positive control) were used. A: GAPDH, 226 bp; B: PRL, 276 bp; C: Pit-I, 304 bp; D: GH, 161 bp; E: GATA-2, 163 bp. Lane 1: Marker VI (Roche); Lane 2: Normal human liver; Lane 3: Normal human pituitary; Lane 4: Pituitary adenoma No. 1 Adherent cells; Lane 5: Pituitary adenoma No. 1 Stem cells; Lane 6: Pituitary adenoma No. 2 Adherent cells; Lane 7: Pituitary adenoma No. 2 Stem cells; Lane 8: Pituitary adenoma No. 3 Adherent cells; Lane 9: Pituitary adenoma No. 3 Stem cells; Lane 10: Blank. Primer sequences and annealing temperatures are reported in Table 2.

FIG. 6 depicts growth hormone concentration in pituitary adenoma stem cell derived tumor in mouse brain in accordance with an embodiment of the present invention. Six weeks after the injection, one mouse from each group was sacrificed and the brain rapidly removed. Under a dissection microscope, the injection area was cut and homogenized in sterile PBS 1×. The ELISA immunoassay was performed in the supernatant fraction of the homogenate. As a control, a tumor from U87 injected mice was used and processed as described herein.

FIG. 7 depicts immunohistochemistry of mouse brain sections in accordance with an embodiment of the present invention. (A) Hematoxylin & eosin staining of a brain section from a mouse injected with tumor stem cells derived from a somatotroph pituitary adenoma. (B) Staining with growth hormone antibody showed positive labeling of injected cells.

FIG. 8 depicts primary pituitary adenoma cells cultured in defined neural stem cell medium with growth factors in accordance with various embodiments of the present invention. Sphere-growing cells can be observed in the primary cells after 7-14 days culture. In the cultures, some areas were growing monolayer cells (a & b). However, sphere-forming cells were also observed in the cultures (c & d). These spheres were morphologically similar to cancer stem cell spheres in human glioblastoma cultures. The sphere-growing cells in the culture became free-floating spheres as the culture continued (e & f). The free-floating spheres were passaged in defined neural stem cell culture media for more than 30 passages without morphological and cell doubling-time changes. The free-floating spheres formed sub-spheres after dissociating into single cells (g & h). The figures shown on the left panel were from pituitary adenoma No. 2, which was a null-cell macroadenoma (a, c, e & g). The figures shown on the right panel were from pituitary adenoma No. 3, which was a somatotroph GH-positive adenoma (b, d, f & h).

FIG. 9 depicts the self-renewal ability of the tumor spheres analyzed by sub-sphere assay in accordance with various embodiments of the present invention. The sub-sphere forming efficiency was quantified in different passages of the tumor spheres.

FIG. 10 depicts sub-spheres formed from single mother cell (from pituitary adenoma No. 2) of tumor spheres expressing stem cell markers and producing hormones in accordance with various embodiments of the present invention. The sub-spheres expressed stem cell marker genes. Nestin positive spheres were observed as stained in green (a & c). Also, CD133 positive spheres were demonstrated as red (d & f). DAPI were used to localize cell nuclei (b & e). The overlay images are also shown (c & f).

FIG. 11 depicts sub-spheres formed from single mother cell (from pituitary adenoma No. 3) of tumor spheres expressing stem cell markers and producing hormones in accordance with various embodiments of the present invention. The sub-spheres expressed stem cell marker genes. Nestin positive spheres were observed as stained in green (a & c). Also CD133 positive spheres were demonstrated as red (d & f). Some growth hormone positive cells were identified around the negative stained tumor spheres (h & j). DAPI were used to localize cell nuclei (b & e). The overlay images are also shown (c, f & j).

FIG. 12 depicts pituitary tumor stem cells forming spheres resembling neurospheres in accordance with various embodiments of the present invention. Tumor spheres from two pituitary tumors (A and B) are shown.

FIG. 13 depicts adherent pituitary tumor cells differentiated from pituitary tumor stem cells in accordance with various embodiments of the present invention. Tumor spheres were switched to differentiation medium and grown for 7-10 days. Two clones (A and B) are shown.

FIG. 14 depicts hormone production by pituitary tumor spheres (open bars) and differentiated cells (closed bars) stimulated with hypothalamus hormones in accordance with various embodiments of the present invention. Upon stimulation with GH-releasing factor (GHRF), PRL-releasing peptide (PRP), and Thyrotropin-Releasing Hormone (TRH) for 24 h, the secretion of GH, PRL, and TSH, respectively, by the differentiated pituitary tumor cells were determined using ELISA. ** p<0.01.

FIG. 15 depicts relative expression of pituitary-lineage transcription factors in pituitary tumor stem cells compared to that of differentiated pituitary tumor cells in accordance with various embodiments of the present invention. mRNA expression levels were determined by reverse transcription followed by quantitative PCR.

FIG. 16 depicts several stem cell related genes in accordance with various embodiments of the present invention. PTCH1, BMI1, GLi1, SOX2, NCAM and Oct4 were highly expressed on pituitary adenoma No. 3 derived clone 1 tumor stem cells and clone 2 tumor stem cells than those on their differentiated cells by real-time PCR analysis.

FIG. 17 depicts tumor spheres' ability to form new tumors in in vivo environments in accordance with various embodiments of the present invention. Tumor spheres can form new tumors upon intracranial implantation into NOD/SCID mice. The tumor-forming ability was confirmed by serial in vivo transplantations. Murine brain sections were immunofluorescence stained with human specific nuclei antibody (green) and growth hormone antibody (red). Human specific stained cells were visualized by FITC-conjugated secondary antibody (green). The growth hormone positive cells were identified by Tex-Red-conjugated secondary antibody (red). DAPI was used for identifying nuclei (blue). The overlay images are also shown.

FIG. 18 depicts the stem cell's ability to form new tumors through serial transplantation in accordance with various embodiments of the present invention. Mice brain sections were immunofluorescence stained with human specific nuclei antibody (green) and growth hormone antibody (red). Human specific positive cells were visualized by FITC-conjugated secondary antibody (green). The growth hormone positive cells were identified by Tex-Red-conjugated secondary antibody (red). DAPI was used for identifying nuclei (blue). The overlay images were shown as well.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Pituitary conditions,” “pituitary-related conditions,” “pituitary disease conditions” and “pituitary-related disease conditions” as used herein may include, but are in no way limited to, any condition or disease condition caused by or related to abnormally functioning pituitary cells, tissues or glands. Examples include, but are not limited to, benign pituitary adenomas, invasive pituitary adenomas, pituitary carcinomas, amenorrhea, galactorrhea, infertility, hypogonadism, gigantism, acromegaly, Cushing's disease and hyperthyroidism.

“Pituitary adenoma” as used herein refers to benign or invasive pituitary adenomas. Examples of pituitary adenomas include, but are not limited to: prolactinomas, somatotrophic adenomas, gonadrotophic adenomas, adrenocorticotropic hormone (ACTH)-secreting adenomas, thyrotropic adenomas and null cell adenomas.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Stem cell” as used herein refers to a cell that can continuously produce unaltered daughters and also has the ability to produce daughter cells that have different, more restricted properties.

“Tumor,” as used herein refers to all neoplastic cell growth and proliferation, whether malignant or benign, and all pre-cancerous and cancerous cells and tissues.

The inventor has, for the first time in humans, isolated and generated pituitary adenoma stem cells and stem cell lines and pituitary adenoma cells and cell lines. Pituitary adenomas are benign tumors and benign cells generally do not grow in culture. However, the inventor believed that pituitary adenomas may contain stem cells. As such, experiments were carried out as described herein and the inventor has identified and isolated tumor stem cells from pituitary adenomas. The inventor has confirmed that the isolated cells are indeed stem cells. With the isolation of these stem cells, the pituitary adenoma cells possess the ability to grow in culture.

Embodiments of the present invention include methods for isolation of pituitary adenoma stem cells, pituitary adenoma cells, pituitary carcinoma stem cells, and pituitary carcinoma cells; and methods for generating pituitary adenoma stem cell lines, pituitary adenoma cell lines, pituitary carcinoma stem cell lines and pituitary carcinoma cell lines. Additional embodiments of the present invention include methods for using pituitary adenoma stem cells or cell lines, pituitary adenoma cells or cell lines, pituitary carcinoma stem cells or cell lines, and pituitary carcinoma cells and cell lines for a variety of purposes; for example, to test therapeutic products, to study pituitary diseases (e.g., pituitary adenomas including but not limited to prolactinomas, somatotrophic adenomas, adrenocorticotropic hormone (ACTH)-secreting adenomas, gonadrotophic adenomas, thyrotropic adenomas and null cell adenomas; pituitary carcinomas; invasive pituitary adenomas; amenorrhea; galactorrhea; infertility; hypogonadism; gigantism; acromegaly; Cushing's disease; and hyperthyroidism), and to obtain human pituitary hormone products (e.g., prolactin, human growth hormone, adrenocorticotropic hormone, and sexual hormones such as luteinizing hormone and follicle-stimulating hormone). All of these uses were previously impossible because no one had heretofore been able to generate a stable human cell line from a pituitary adenoma.

Isolation and generation of pituitary adenoma stem cells and cell lines, pituitary adenoma cells and cell lines, pituitary carcinoma stem cells and cell lines, and pituitary carcinoma cells or cell lines may be accomplished as described by Yuan et al., “Isolation of cancer stem cells from adult glioblastoma multiforme,” Oncogene (2004) 23; 9392-9400, hereby incorporated by reference as though fully set forth in its entirety. In one embodiment, tumor samples may be collected from surgical resection. Tissues may be washed and dissected in a dissection medium containing catalase, deferoxamine, N-Acetyl cysteine and superoxide dismutase. After digestion in trypsin for about 10 min at about 37° C., tumor tissues may be triturated. Tituration may be performed by passing the tissues in a tissue sieve and after recovering the cells by passing them through a 70 pm cell strainer. Cells may then plated at the density of about 1×10⁵ cells/ml in a medium containing DMEM/F12 (1:1) (Gibco), 10% FBS (Omega Scientific), penicillin/streptomycin (200 U/ml; Gibco) and Glutamax 1× (Gibco). After about 24-48 hour, medium may be changed with a medium containing DMEM/F12 (1:1) (Gibco), B27 1× (Gibco), penicillin/streptomycin (200 U/ml; Gibco), fungizone (250 ng/ml), EGF (20 ng/ml) and bFGF (20 ng/ml). For each cell line, daughter cells growing adherent in 10% FBS containing medium may be prepared.

The pituitary adenoma stem cells or cell lines, pituitary adenoma cells or cell lines, pituitary carcinoma stem cells or cell lines, and pituitary carcinoma cells or cell lines of the present invention may be used to test therapeutic products and to discover and develop novel anti-tumor and anti-cancer therapies. Several types of experimental approaches can utilize these cell lines in vitro and in vivo, including conventional chemotherapies, immunomodulatory therapies, and gene therapies affecting pituitary cells. Examples of these therapies include but are not limited to: cytostatic agents, biological response modifiers, cytokine expressing agents, gene therapy vector agents, immunotoxin agents, antiproliferative agents, anti-metastasis agents, and angiostatic agents.

In various embodiments, the pituitary adenoma stem cells or cell lines, or pituitary carcinoma stem cells or cell lines may be used for identifying compounds or conditions that induce or inhibit differentiation of pituitary cells. The pituitary adenoma stem cells or cell lines, and the pituitary carcinoma stem cells or cell lines may be useful for screening chemical agents to identify chemicals which may induce or inhibit pituitary adenoma, pituitary carcinoma or other related diseases in vitro. To determine whether a chemical can induce or inhibit differentiation, pituitary adenoma stem cells or pituitary carcinoma stem cells may be cultured; for example, by plating a tissue culture plate in a medium at about 37° C. In particular embodiments, a test compound may be added to the cells with each medium change. At particular time points, the ability of the test compound to induce or inhibit differentiation of the cells may be determined.

In another embodiment, the pituitary adenoma stem cells or cell lines, pituitary adenoma cells or cell lines, pituitary carcinoma stem cells or cell lines, and pituitary carcinoma cells or cell lines may be useful in methods for determining whether a compound (e.g., a chemotherapeutic agent, an antiproliferation agent, a cytotoxic agent, etc.) or particular culture conditions can induce proliferation or inhibit proliferation of pituitary adenoma stem cells, pituitary adenoma cells, pituitary carcinoma stem cells, or pituitary carcinoma cells. Particular compounds may induce the pituitary adenoma stem cells, pituitary adenoma cells, pituitary carcinoma stem cells, or pituitary carcinoma cells to proliferate. To determine the ability of a compound to induce proliferation in pituitary cells, pituitary adenoma stem cells, pituitary adenoma cells, pituitary carcinoma stem cells or pituitary carcinoma cells may be cultured. A test compound may be added to the cells with each medium change. At particular time points, the effect of the test compound on the cells may be determined.

In accordance with another embodiment of the invention, the pituitary adenoma stem cells or cell lines, pituitary adenoma cells or cell lines, pituitary carcinoma stem cells or cell lines, or pituitary carcinoma cells or cell lines may be used to screen for therapeutic compounds as evidenced by a test compound's ability to modulate a biochemical activity of the cells (e.g., the cells' growth, signaling pathways, etc.).

In one exemplary application of the screening method, the cells are grown in a suitable medium and a test compound is added to the culture to determine the effect on the cells. For example, a suspension of cultured cells may be aliquoted into each of several wells, and increasing amounts of the test compound, (e.g., 0, 10, 100, 1000, 10,000 mM) are added to the wells. After a suitable incubation time, the level of the detectable marker protein in the wells is measured to determine if the compound, at any concentration, has resulted in affecting the cell.

Compounds tested may include known anti-tumor compounds. Compounds identified as anti-tumor compound candidates may be further tested in defined screening systems, such as animal model systems, to further assess the potential of the compound as an anti-tumor agent.

It will be appreciated that the screening format is readily adaptable to high throughput screening (HIS), for example, by simultaneously screening a large number of samples in the microtiter wells of a multiwell plate, such as one having 96, 720 or larger numbers of wells. The wells may be readily assayed for a compound's effect, simply by assaying the level of the fluorescence from the cell samples at optimal fluorescence excitation and emission wavelengths. Compounds that test positive may then be retested for more precise dose response to further determine the potential value of the compound.

When a test compound modulates the level of the detectable marker and/or its activity at a pharmaceutically practical level has been identified, the compound may be further assayed to develop its pharmacological profile. Such tests may include in vitro cell-culture studies to determine the effect of the identified compound, the ability of the identified compound to inhibit proliferation, the ability of the compound to inhibit proliferation in suitable animal model systems, and the toxicology profile of the compound in animals.

In addition, when test compounds are identified, the compound may be further developed by standard drug-design or combinatorial-structure approaches to seek more active analogs, and/or compounds with reduced toxicity.

Other embodiments of the present invention utilize the pituitary adenoma stem cells or cell lines, pituitary adenoma cells or cell lines, pituitary carcinoma stem cells or cell lines, or pituitary carcinoma cells or cell lines to study pituitary diseases. Examples of pituitary diseases include but are not limited to pituitary adenomas (e.g., prolactinomas, somatotrophic adenomas, adrenocorticotropic hormone (ACTH)-secreting adenomas, gonadrotophic adenomas, thyrotropic adenomas and null cell adenomas), amenorrhea, galactorrhea, infertility, hypogonadism, gigantism, acromegaly, Cushing's disease, and hyperthyroidism. For example, the stem cells or adherent cells described herein may be implanted into laboratory animals (e.g., mice, rat, etc.) for various in vivo studies. These studies may investigate the genetic or other biological etiologies of pituitary adenomas or pituitary carcinomas. Other studies may test compounds or therapies for the treatment of pituitary adenomas, pituitary carcinomas, pituitary conditions or disease conditions and/or pituitary-related conditions or disease conditions.

Additional embodiments of the present invention may utilize pituitary adenoma stem cells or cell lines, pituitary adenoma cells or cell lines, pituitary carcinoma stem cells or cell lines, or pituitary carcinoma cells or cell lines to obtain human pituitary hormone products (e.g., prolactin, human growth hormone, adrenocorticotropic hormone, and sexual hormones such as luteinizing hormone and follicle-stimulating hormone). Cells lines as described herein may be used to produce hormone products. The hormone products may be isolated from the media or from the intracellular contents.

The present invention is also directed to kits for isolating pituitary adenoma stem cells, pituitary adenoma cells, pituitary carcinoma stem cells, and pituitary carcinoma cells; kits for generating pituitary adenoma stem cell lines, pituitary adenoma cell lines, pituitary carcinoma stem cell lines, and pituitary carcinoma cell lines; and kits for using pituitary adenoma stem cells or cell lines, pituitary adenoma cells or cell lines, pituitary carcinoma stem cells or cell lines, or pituitary carcinoma cells or cell lines to test therapeutic products, to study pituitary conditions or disease conditions, to study pituitary-related conditions or disease conditions and/or to obtain human pituitary hormone products.

Each kit is an assemblage of materials or components. The exact nature of the components configured in each inventive kit depends on its intended purpose. For example, some embodiments are configured for the purpose of isolating the pituitary stem cells and/or cells described herein; generating the pituitary stem cell lines and/or cell lines described herein; and/or using the pituitary stem cells and stem cell lines or the cells or cell lines described herein to test therapeutic products, to study pituitary adenomas, pituitary carcinomas, pituitary conditions or disease conditions and pituitary-related conditions or disease conditions, and/or to obtain human pituitary hormone products. In some embodiments, the kits are configured particularly for mammalian subjects. In another embodiment, the kits are configured particularly for human subjects. In further embodiments, the kits are configured for veterinary animals, such as such as, but not limited to, farm animals, domestic animals, and laboratory animals.

Instructions for use may be included in the kit. “Instructions for use” typically include a tangible expression describing the technique to be employed in using the components of the kit for a desired purpose, such as for isolating pituitary adenoma stem cells, pituitary adenoma cells, or pituitary carcinoma cells; generating pituitary adenoma stem cell lines, pituitary adenoma cell lines, pituitary carcinoma stem cell lines, or pituitary carcinoma cell lines; and/or using pituitary adenoma stem cell lines, pituitary adenoma cell lines, pituitary carcinoma stem cell lines, or pituitary carcinoma cell lines to test therapeutic products, to study pituitary adenomas or carcinomas, to study pituitary conditions or disease conditions, to study pituitary-related conditions or disease conditions and/or to obtain human pituitary hormone products. Optionally, the kits also contain other useful components, such as, buffers (e.g., PBS), growth media, tissue culture plates, multiple-well plates, flasks, chamber slides, differentiation media, stem cell media, tumor stem cell media, cancer stem cell media, neural stem cell media, goat serum, fetal bovine serum, basic fibroblast growth factor, epidermal growth factor, diluents, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a cryocontainer used to contain suitable quantities of pituitary stem cells and/or pituitary cells described herein. The packaging material generally has an external label which indicates the contents and/or purpose of the kit and/or its components.

One skilled in the art will recognize many methods and materials similar or equivalent to those described herein, which could be used in the practice of the present invention. Indeed, the present invention is in no way limited to the methods and materials described.

### Examples

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Patients

Tumor stem neurospheres were prepared from four pituitary macroadenomas null-cell, two somatotroph (growth hormone producing) pituitary adenomas and two pituitary macroadenomas with acromegaly (see Table 1).

TABLE 1
Tumor and patients characteristics

| Tumor | Patient age | Diagnosis |
|---|---|---|
| PA 1 | 43 | microadenoma GH+ |
| PA 2 | 58 | macroadenoma null-cell |
| PA 3 | 19 | somatotroph GH+ |
| PA 4 | 40 | macroadenoma with |
| PA 5 | 86 | macroadenoma null-cell |
| PA 6 | 64 | macroadenoma null-cell |
| PA 7 | 57 | adenoma with acromegaly |
| PA 8 | 50 | macroadenoma null-cell |

### Example 2

### Preparation of Tumor Stem Neurospheres from Pituitary Adenomas

Tumor stem neurospheres were prepared as described by Yuan et al. Briefly, tumor samples from eight pituitary adenoma patients were collected within half an hour from the surgical resection as approved by the Institutional Review Board at Cedars Sinai Medical Center. Tissues were washed three times in PBS 1× and dissected in a dissection medium containing catalase, deferoxamine, N-Acetyl cysteine and superoxide dismutase. After digestion in trypsin for 10 min at 37° C., tumor tissues were triturated by passing them in a tissue sieve and after recovering the cells by passing in a 70 μm cell strainer. Cells were then plated at the density of 1×10⁵ cells/ml in a medium containing DMEM/F12 (1:1) (Gibco), 10% FBS (Omega Scientific), penicillin/streptomycin (200 U/ml; Gibco) and Glutamax 1× (Gibco). After 24-48 h medium was changed with a medium containing DMEM/F12 (1:1) (Gibco), B27 1× (Gibco), penicillin/streptomycin (200 U/ml; Gibco), fungizone (250 ng/ml), EGF (20 ng/ml) and bFGF (20 ng/ml). For each cell line, daughter cells growing adherent in 10% FBS containing medium were prepared.

### Example 3

### Immunocytochemistry Analysis of Tumor Stem Neurospheres Derived from Pituitary Tumors

Neurospheres from pituitary tumors were analyzed by immunocytochemistry after growing them in poly-lysine coated chamber slides with or without growth factors for 10 days (differentiation conditions). Cells were fixed in PBS 1×/4% paraformaldehyde, and after several washing with PBS 1×, they were permeabilized in 0.1% Triton X-100 and blocked with 10% goat serum in PBS 1×. Cells were then incubated with primary antibodies: GFAP (1:200, Dako, Denmark), β-III Tubulin (1:400, Covance, Berkeley, Calif.), S-100 (1:200, Chemicon), CNPase (1:200), nestin (1:50, Chemicon, Temecula, Calif.) and CD133 (1:200, Milteny Biotec, Auburn, Calif.). After incubation with FITC conjugated secondary antibodies (1:300), slides were counter-stained with a mounting medium containing DAPI (Vector Laboratories) before examination by fluorescence microscopy.

### Example 4

### Determination of Pituitary Hormones Production from Tumor Stem Neurospheres Derived from Pituitary Adenomas

In order to determine and quantify the pituitary hormones production from the tumor stem cells derived from the pituitary tumors, 5×10⁵ cells from each cell line were plated in 5 wells of a six-wells plate. For each cell line, the daughter adherent cells were plated in parallel in other plates. Conditioned medium from each cell line was collected 2, 4, 8, 16 and 24 h after the plating and kept at −80° C. until use. ELISA kits (GH, PRL, FSH, LH, TSH were from Anogen, ACTH from R&D Systems) were used for the quantification of the pituitary hormones and the suggested protocols were followed. Quantification of growth hormone and prolactin was also performed in brain homogenates from mice-injected with pituitary adenoma cancer stem cells.

### Example 5

### In Vivo Injection of Pituitary Adenoma Tumor Stem Neurospheres

Pituitary adenomas neurospheres (1×10⁴) and adherent daughter cells (1×10⁵) from one null and one growth hormone-producing tumor were stereotactically injected into the right hemisphere (coordinates from Bregma: 0.5 mm anterior, 2.2 mm right, 3.0 depth) of NOD/SCID mice (The Jackson Laboratories, Bar Harbor, Me., USA; n=4 for each group). Control mice (n=3) were injected with 1×10⁴ U87 glioma cell line. The experiment was repeated once with identical conditions. Mice were sacrificed at different time points and brain sections of the tumor were examined as described below. All the experiments were performed in the accordance with the Institutional Animal Care and Use Committee guidelines from Cedars Sinai Medical Center.

### Example 6

### Immunohistochemistry Staining of Brain Sections

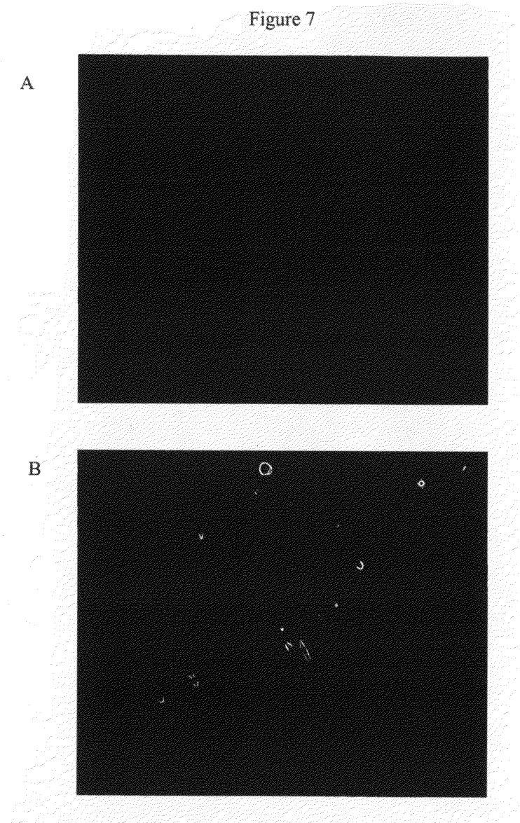

Brains from injected mice were post fixed with 10% buffered formalin at 4° C. After inclusion in paraffin, brains were cut, mounted on slides and stained with hematoxylineosin. Immunohistochemical analysis was also performed to stain for the pituitary hormones as the indicated concentrations: growth hormone (1:1000; Dako), prolactin (1:1500; Dako); follicle-stimulating hormone (FSH, 1:200; Dako), luteinizing hormone (LH, 1:2000; Dako), adrenocorticotropic hormone (ACTH, 1:400; Dako), thyroid-stimulating hormone (TSH, 1:100; Dako), alpha-subunit (1:8000: Abcam). Hematoxylin & eosin staining of a brain section from a mouse injected with pituitary adenoma stem cells derived from a somatotroph pituitary adenoma was performed. (FIG. 7A.) Additionally, staining with growth hormone antibody also showed positive labeling of injected cells. (FIG. 7B.)

### Example 7

### RNA Extraction and RT-PCR Assay

RNA was extracted from pituitary adenoma stem cells and adherent cells using the Absolutely RNA miniprep kit (Statagene) following the suggested protocol. After spectrophotometer quantification two micrograms from each sample were subjected to reverse transcription using SuperScript™ II Reverse Transcriptase (Invitrogen) and Random primers (Roche). As controls, RNA from normal human liver (negative control) and normal human pituitary (positive control) were used (BioChain). The PCRs were performed in a 50 μl reaction mixture containing 2 μl cDNA as template and specific oligonucleotides listed in Table 2. Annealing temperatures and sizes of the amplification products are reported in Table 2.

TABLE 2
Oligonucleotide sequences. In the table sequences
of primers used, annealing temperature and
amplicon size are reported.

| | | Annealing | |
|---|---|---|---|
| Primer name | Sequence | temperature | Amplication |
| LH Fw | 5′-GCCATCCTGGCTGTCGAGAAG-3′ | 60° C. | 292 bp |
| | (SEQ ID NO. 1) | | |
| LH Rev | 5′-GAGCCGGATGGACTCGAAGCG-3′ | | |
| | (SEQ ID NO. 2) | | |
| TSH Fw | 5′-ACAATGCACATCGAAAGGAGA-3′ | 60° C. | 238 bp |
| | (SEQ ID NO. 3) | | |
| TSH Rev | 5′-TCCTGGTATTTCTACAGTCCT-3′ | | |
| | (SEQ ID NO. 4) | | |
| FSH Fw | 5′-ATAGAGAAAGAAGAATGTCGT-3′ | 55° C. | 172 bp |
| | (SEQ ID NO. 5) | | |
| FSH Rev | 5′-GTGAGCACAGCCGGGCACTCT-3′ | | |
| | (SEQ ID NO. 6) | | |
| ACTH Fw | 5′-AGCTTGGCCATATCTGATATG-3′ | 60° C. | 261 bp |
| | (SEQ ID NO. 7) | | |
| ACTH Rev | 5′-GATGTAGCGGTCCGCAGCAAT-3′ | | |
| | (SEQ ID NO. 8) | | |
| GH Fw | 5′-ATGACACCTATCAGGAGTTTGAAGAAG-3′ | 58° C. | 161 bp |
| | (SEQ ID NO. 9) | | |
| GH Rev | 5′-GATGCGGAGCAGCTCTAGGTTAGATTT-3′ | | |
| | (SEQ ID NO. 10) | | |
| PRL Fw | 5′-GGGTTCATTACCAAGGCCATCA-3′ | 58° C. | 276 bp |
| | (SEQ ID NO. 11) | | |
| PRL Rev | 5′-TTCAGGATGAACCTGGCTGAC-3′ | | |
| | (SEQ ID NO. 12) | | |
| PROP1 Fw | 5′-GAGTCAGCCTTTGGGAGGAAC-3′ | 58° C. | 237 bp |
| | (SEQ ID NO. 13) | | |
| PROP1Rev | 5′-TGGTGGTGGTGGTGCTGCGTA-3′ | | |
| | (SEQ ID NO. 14) | | |
| Pit1 Fw | 5′-ACAGCTGCTGATTTCAAGCA-3′ | 56° C. | 304 bp |
| | (SEQ ID NO. 15) | | |
| Pit1 Rev | 5′-ACAAAGCTCCTACTTGCTCA-3′ | | |
| | (SEQ ID NO. 16) | | |
| GATA-2 Fw | 5′-CCCTAAGCAGCGCAGCAAGAC-3′ | 61° C. | 163 bp |
| | (SEQ ID NO. 17) | | |
| GATA-2 Rev | 5′-GATGAGTGGTCGGTTCTGGCC-3′ | | |
| | (SEQ ID NO. 18) | | |
| Alpha-subunit | 5′-TCCGCTCCTGATGTGCAGGAT-3′ | 58° C. | 132 bp |
| Fw | (SEQ ID NO. 19) | | |
| Alpha-subunit | 5′-GGACCTTAGTGGAGTGGGATA-3′ | | |
| Rev | (SEQ ID NO. 20) | | |
| GAPDH FW | 5′-GAAGGTGAAGGTCGGAGT-3′ | 54° C. | 226 bp |
| | (SEQ ID NO. 21) | | |
| GAPDH Rev | 5′-GAAGATGGTGATGGGATTTC-3′ | | |
| | (SEQ ID NO. 22) | | |

### Example 8

### Clonal Analysis

Cells from the pituitary adenoma were plated at a low density (1000 cells/1 ml of complete medium containing EGF/bFGF) on a 96-well plate. The presence of single spheres was checked.

### Example 9

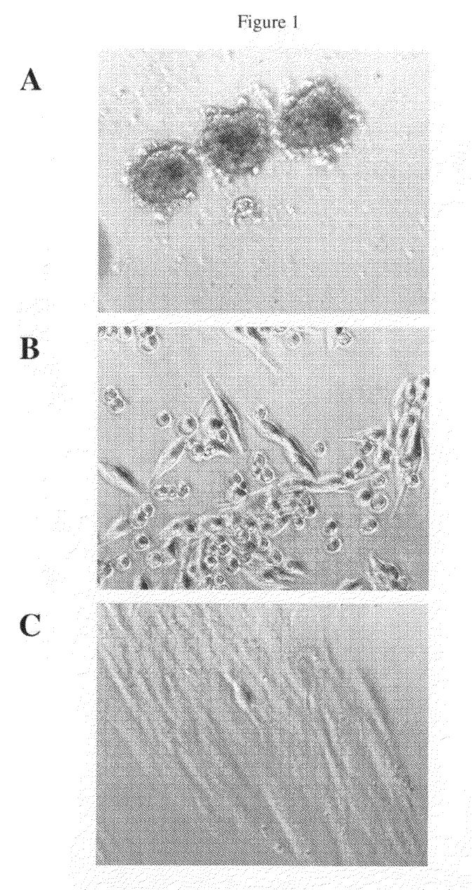

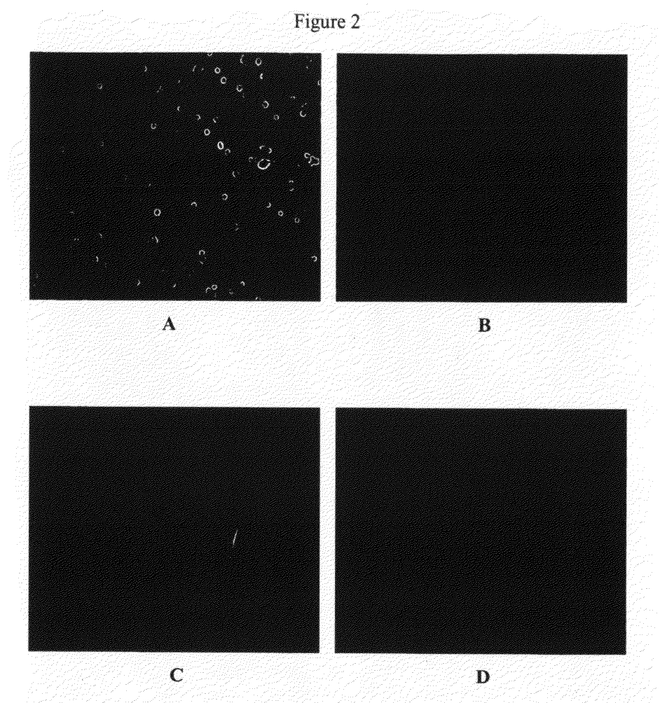

Pituitary adenomas cells growing in a medium containing EGF and bFGF showed the formation of spheres, characteristic of stem cells (FIG. 1A). The corresponding adherent daughter cells have a typical fibroblast-like phenotype (FIG. 1C). When spheres derived from these pituitary adenomas were subjected to differentiation conditions (withdrawal of growth factors and plating onto poly-lysine coated slides, FIG. 1B), these cells showed staining for nestin (FIG. 2A), β-III tubulin (FIG. 2C) and GFAP (FIG. 2E). In addition they showed staining for S-100, a protein reported to be present in stellate cells of the pars distalis and tuberalis, in the marginal cells and in pituicytes of the neural lobe of pituitary.

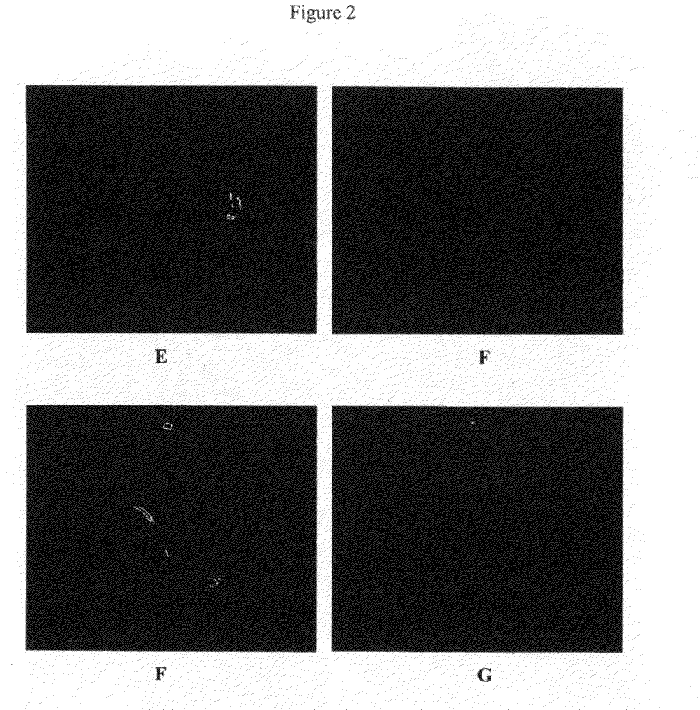

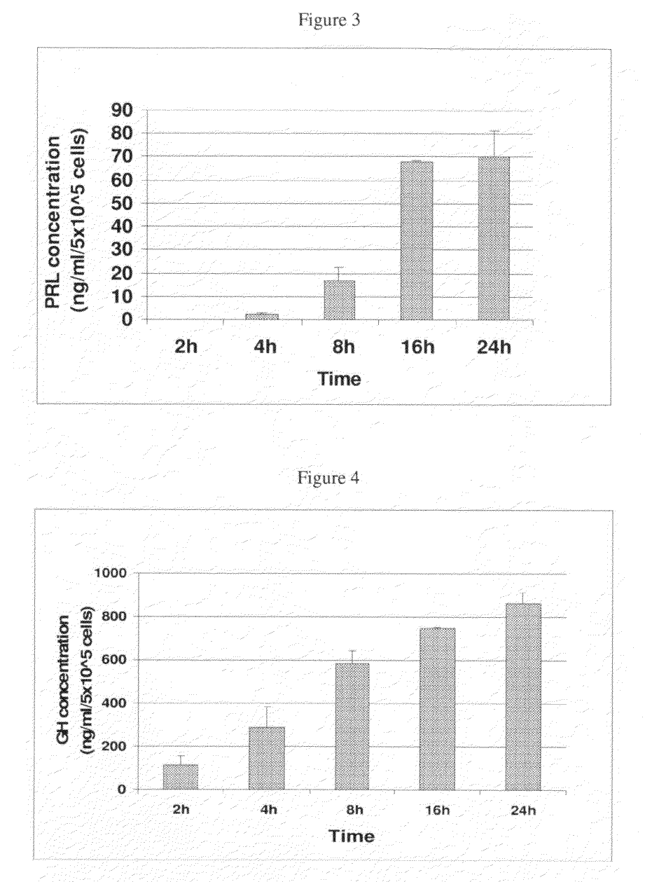

ELISA quantification of the hormone production showed a higher production of prolactin and growth hormone in one tumor stem cell line derived from a patient with a somatotroph adenoma, while the adherent counterpart was not hormone producing (FIG. 3 and FIG. 4). The levels of the other hormones were also investigated and they remain under normal values (data not shown).

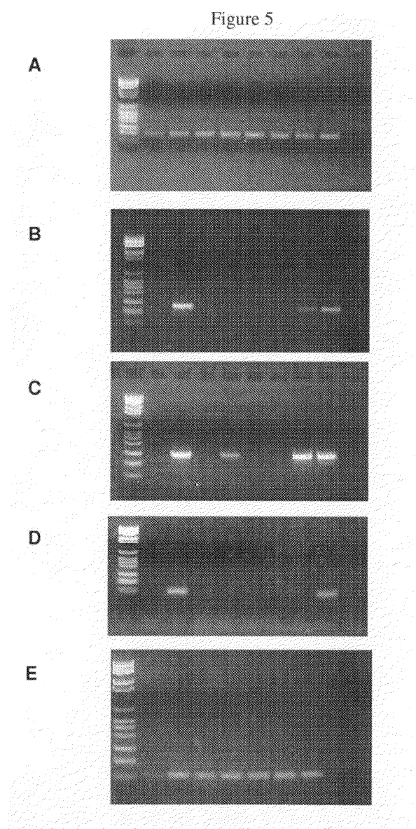

With semi-quantitative RT-PCT the inventor also detected the levels of expression of all the pituitary hormones as well as that of pituitary-related transcription factors. In particular, the inventor found that the pituitary specific transcription factor Pit-1 is expressed in two of three tumor stem cells analyzed. In one pituitary cell line, GATA-2, a zinc finger transcription factor necessary for differentiation and determination of gonadotrophs and thyrotrophs, is present only in adherent cells and not in tumor stem cells (FIG. 5).

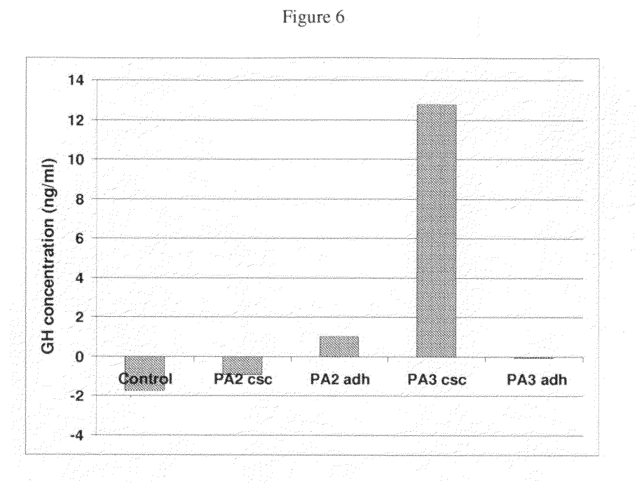

Pituitary adenomas stem derived cells were also injected in vivo in the right striatum of NOD/SCID mice. Six week after the injection part of the animals were sacrificed for histology and immunohistochemistry analysis and for the detection of growth hormone and prolactin in the mouse-tumor homogenate with ELISA immunoassay. In the brain of a mouse injected with the somatotroph adenoma-derived tumor stem cells, the inventor found the presence of the injected cells that stained positive for growth hormone. By ELISA immunoassay performed in the tumor homogenate from another mouse injected with the same cells, the inventor found a higher percentage of growth hormone with respect to control animals injected with U87 glioma cells and to mice injected with daughter adherent cells (FIG. 6). Taking into account that the ELISA assay used is specific to detect human growth hormone and do not cross react with mouse, this results confirm that these cells survive after the injection and they are able to produce the growth hormone also in vivo into the mouse host brain.

### Example 10

### Pituitary Adenomas Contain Sphere-Growing Cells that Have Self-Renewal Ability

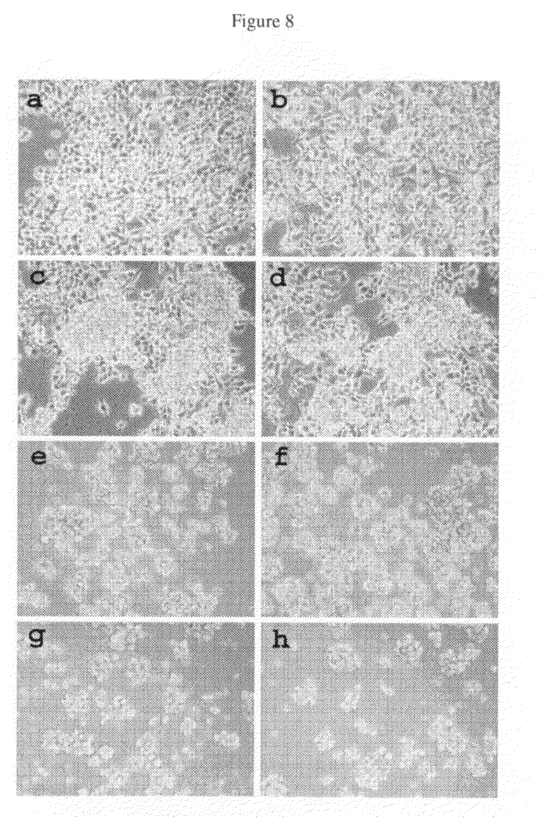

Primary pituitary adenoma cells were cultured in defined neural stem cell medium with growth factors (EGF, 20 ng/ml and bFGF, 20 ng/ml, PeproTech Inc, Rocky Hill, N.J.). Sphere-growing cells were observed in the primary cells after 7-14 days culture. In the cultures, some areas were growing as monolayer cells (a & b). Sphere-forming cells were also observed in the cultures (c & d). These spheres were morphologically similar as cancer stem cell spheres in the human glioblastoma cultures. The sphere-growing cells in the culture became free-floating spheres as the culture continued (e & f). The free-floating spheres were passaged in the defined neural stem cell culture media for more than thirty passages without morphological and cell doubling-time changes. The free-floating spheres formed sub-spheres after dissociating into single cells (g & h). These characteristics indicate the self-renewal ability of the sphere-growing pituitary adenoma cells (see FIG. 8). The images shown on the left panel were from pituitary adenoma No. 2, which is a null-cell macroadenoma (a, c, e & g). The images shown on the right panel were from pituitary adenoma No. 3, which is a somatotroph GH positive adenoma (b, d, f & h).

### Example 11

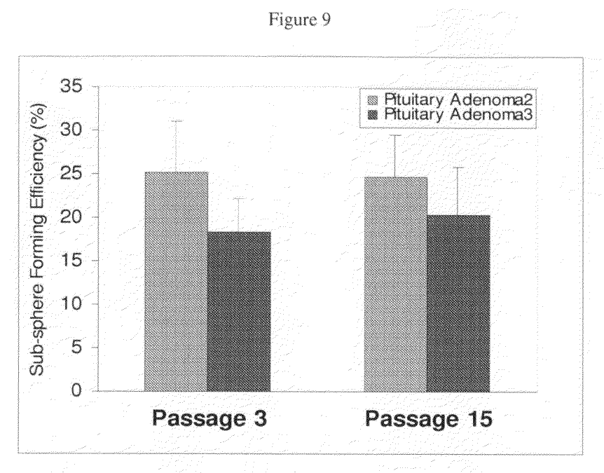

To confirm the self-renewal ability of the adenoma tumor spheres, sub-sphere assay was performed in 96-well culture plates. The spheres were mechanically dissociated into single cells and diluted into culture medium. The suspended cells were seeded into 96-well plates with the dilution that resulted in one cell per well. The wells containing a single cell were identified by checking the culture wells 2 hours post the cell seeding process. After two weeks, culture with medium refreshed every three days, the culture wells were observed and the sub-sphere containing wells were counted. This sub-sphere assay was done with different passages of the sphere cells and experiments were repeated once. (See FIG. 9.)

### Example 12

### Sub-Spheres Formed from Single Mother Cell of Tumor Spheres can Express Stem Cell Markers and Hormones

To study whether the self-renewable spheres expressed stem cell genes, the inventor investigated the expression profile of stem cell markers, nestin and CD133. The single sphere cell-derived sub-spheres can be stained positive for nestin (1:100, Chemicon, Temecula, Calif.) and CD133 (1:200, Abcam, Cambridge, Mass.). The somatotroph growth hormone (GH) positive adenoma derived tumor spheres were also stained for GH (1:2000, Chemicon, Temecula, Calif.). The primary antibodies were visualized by FITC or Tex-Red conjugated secondary antibodies (Jackson ImmunoResearch, West Grove, Pa.). Cell nuclei were stained by DAPI (Invitrogen, Carlsbad, Calif.).

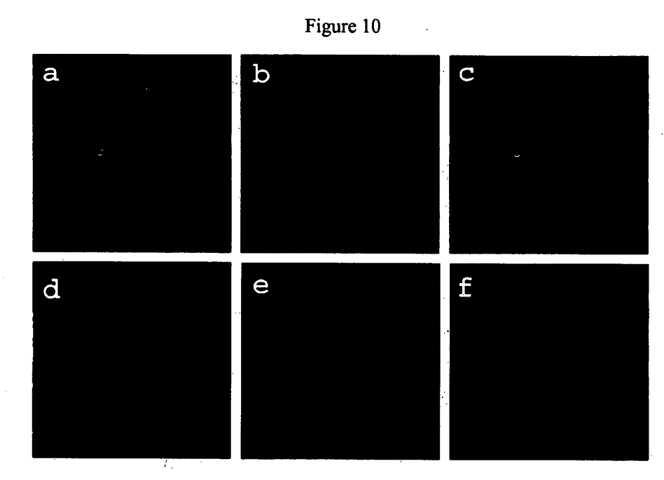

FIG. 10 shows pituitary adenoma No. 2. The sub-spheres expressed stem cell marker genes. Nestin positive spheres were observed as stained in green (a & c). Also, CD133 positive spheres were demonstrated as red (d & f). DAPI were used to localize cell nuclei (b & e). The overlay images are also shown (c & f).

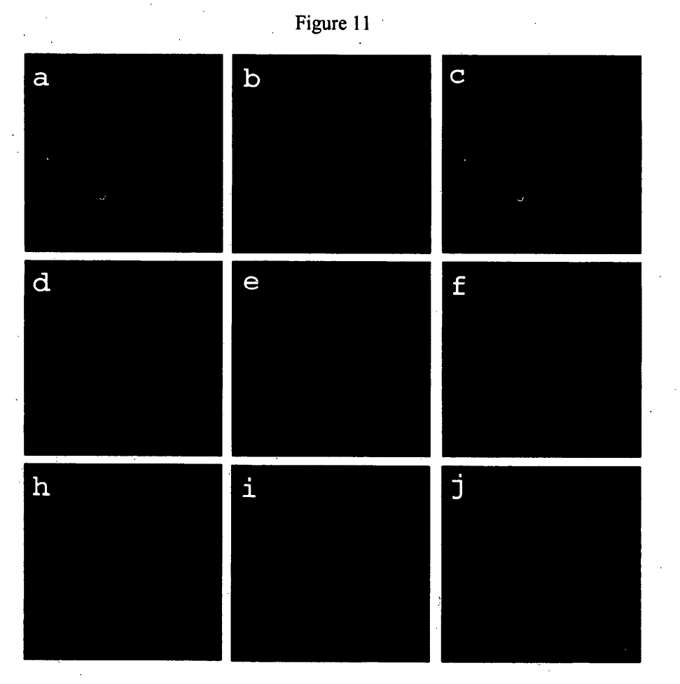

FIG. 11 shows the pituitary adenoma No. 3. The sub-spheres expressed stem cell marker genes. Nestin positive spheres were observed as stained in green (a & c). Also, CD133 positive spheres were demonstrated as red (d & f). Some growth hormone positive cells were identified around the negative stained tumor spheres (h & j). DAPI were used to localize cell nuclei (b, e & i). The overlay images were shown as well (c, f & j).

These data indicate that single sphere cell-derived self-renewable sub-spheres express stem cells markers.

### Example 13

### Single Mother Cell Formed Spheres were Multipotent Upon Differentiation

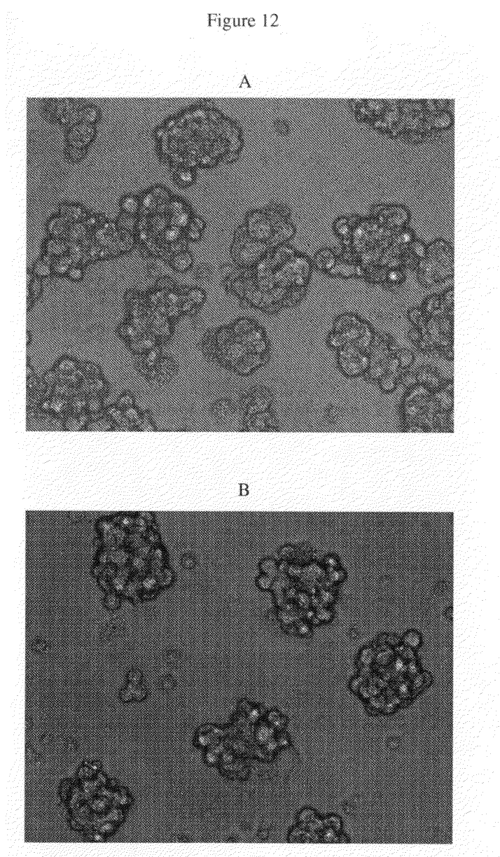

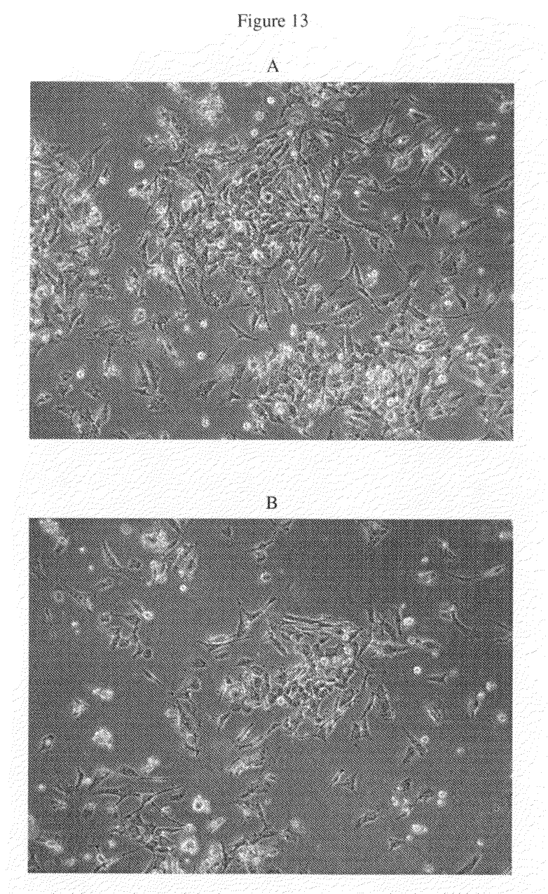

Pituitary tumor stem cells formed spheres resembling neurospheres. Tumor spheres from two pituitary tumors are shown in FIG. 12. Adherent pituitary tumor cells differentiated from the pituitary tumor stem cells. The tumor spheres (stem cells) were switched to differentiation medium and grown for 7-10 days. Two clones are shown in FIG. 13.

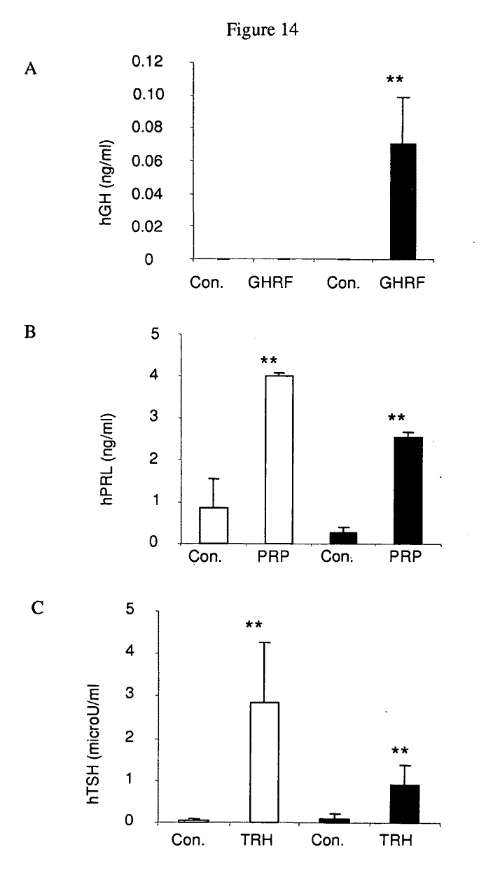

Hormone production by pituitary tumor spheres and differentiated cells was observed. (See FIG. 14.)

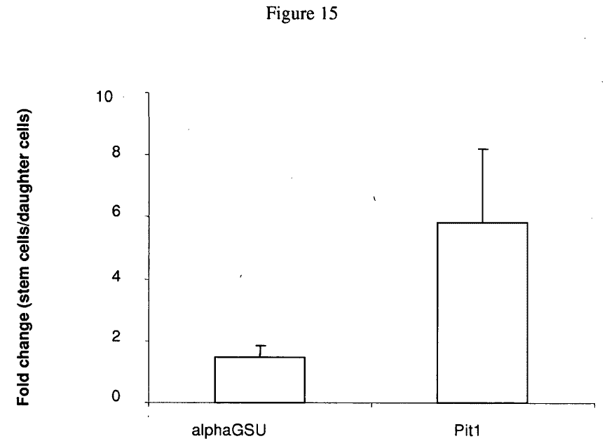

Relative expression of pituitary-lineage transcription factors in pituitary stem cells compared to that of differentiated pituitary tumor cells was also determined. (See FIG. 15.)

### Example 14

### Differentiation of Pituitary Tumor Stem Cells

Pituitary tumor spheres were switched to the differentiation culture medium (DMEM/F12 medium with 2 mM glutamine, 15% horse serum, and 2.5% fetal bovine serum). After the pituitary tumor cells attached to the culture dish, these cells were allowed to grow in the differentiation medium for 7-10 days.

### Example 15

### Stimulated Hormone Production

Pituitary tumor spheres or differentiated pituitary tumor cells were cultured with or without 1×10⁻⁷ M GH-releasing factor (GHRF), 2×10⁻⁷ M PRL-releasing peptide (PRP), and 1×10⁻⁷ M Thyrotropin-Releasing Hormone (TRH) for 24 h. The secreted hormones (GH, PRL, and TSH) in the conditioned media were determined using ELISA kits (Anogen).

### Example 16

### Tumor Spheres had Different Gene Expression Patterns Compared to Their Differentiated Progenies and Monolayer Non-Tumor Cells

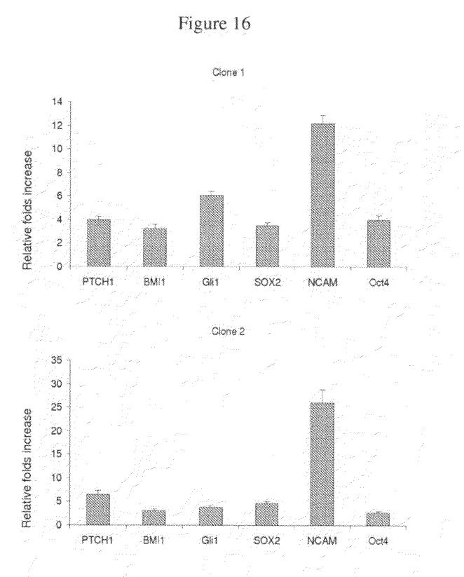

Several stem cell related genes including PTCH1, BMI1, Gli1, SOX2, NCAM and Oct4 were highly expressed on PA No. 3 derived clone 1 tumor stem cells and clone 2 tumor stem cells as compared to those on their differentiated cells. (See FIG. 16.)

### Example 17

### RNA Isolation and cDNA Synthesis

Total RNA was extracted from fresh tumor tissue and isolated CD133 positive and CD133 negative cells using an RNA4PCR kit (Amibion, Austin, Tex.) according to the manufacturer's protocol. For cDNA synthesis, ˜1 μg total RNA was reverse-transcribed into cDNA using Oligo dT primer and iScript cDNA synthesis kit reverse transcriptase. cDNA was stored at −20° C. for PCR.

### Example 18

### Real-Time Quantitative RT-PCR

Gene expression was quantified by real-time quantitative RT-PCR using QuantiTect SYRB Green dye (Qiagen, Valencia, Calif.). DNA amplification was carried out using Icycler (BIO-RAD, Hercules, Calif.), and the detection was performed by measuring the binding of the fluorescence dye SYBR Green I to double-stranded DNA. All the primer sets were provided by Qiagen as shown in Table 3. The relative quantities of target gene mRNA against an internal control, GAPDH, was possible by following a ΔC_{T} method. An amplification plot that had been the plot of fluorescence signal vs. cycle number was drawn. The difference (ΔC_{T}) between the mean values in the duplicated samples of target gene and those of GAPDH were calculated by Microsoft Excel and the relative quantified value (RQV) was expressed as 2^{-ΔC}_{T}. The relative expression of each gene presented in each clone was compared between tumor stem cells versus differentiated cells.

TABLE 3
Oligonucleotide primers sequences used
for SYBR Green real-time PCR

| Primer | | |
|---|---|---|
| name | | Sequence |
| GAPDH | Fw | 5′-CGTCTTCACCACCATGGAGA-3′ |
| | | (SEQ ID NO. 23) |
| | Rev | 5′-CGGCCATCACGCCACAGTTT-3′ |
| | | (SEQ ID NO. 24) |
| GLI1 | Fw | 5′-AGGGAGGAAAGCAGACTGAC-3′ |
| | | (SEQ ID NO. 25) |
| | Rev | 5′-CCAGTCATTTCCACACCACT-3′ |
| | | (SEQ ID NO. 26) |
| PTCH1 | Fw | 5′TGTGATGTGGGAAAGCAGGAGGAT-3′ |
| | | (SEQ ID NO. 27) |
| | Rev | 5′-ACATGTGCTGGTCTCTGGTTACGA-3′ |
| | | (SEQ ID NO. 28) |
| Oct4 | Fw | 5′-CCTGAAGCAGAAGAGGATCA-3′ |
| | | (SEQ ID NO. 29) |
| | Rev | 5′-CCGCAGCTTACACATGTTCT-3′ |
| | | (SEQ ID NO. 30) |
| NCAM | Fw | 5′-AACCAGCAAGGAAAATCCAA-3′ |
| | | (SEQ ID NO. 31) |
| | Rev | 5′-AGGAGCAGGACGAAGATGAC-3′ |
| | | (SEQ ID NO. 32) |
| SOX2 | Fw | 5′-ACCAGCTCGCAGACCTACAT-3′ |
| | | (SEQ ID NO. 33) |
| | Rev | 5′-GTGGGAGGAAGAGGTAACCA-3′ |
| | | (SEQ ID NO. 34) |
| BMI1 | Fw | 5′-GGAGACCAGCAAGTATTGTCCTTTTG-3′ |
| | | (SEQ ID NO. 35) |

| | |
|---|---|
| Rev | 5′-CATTGCTGCTGGGCATCGTAAG-3′ |
| | (SEQ ID NO. 36) |

### Example 19

### Tumor Spheres can Form New Tumors Upon Intracranial Implantation

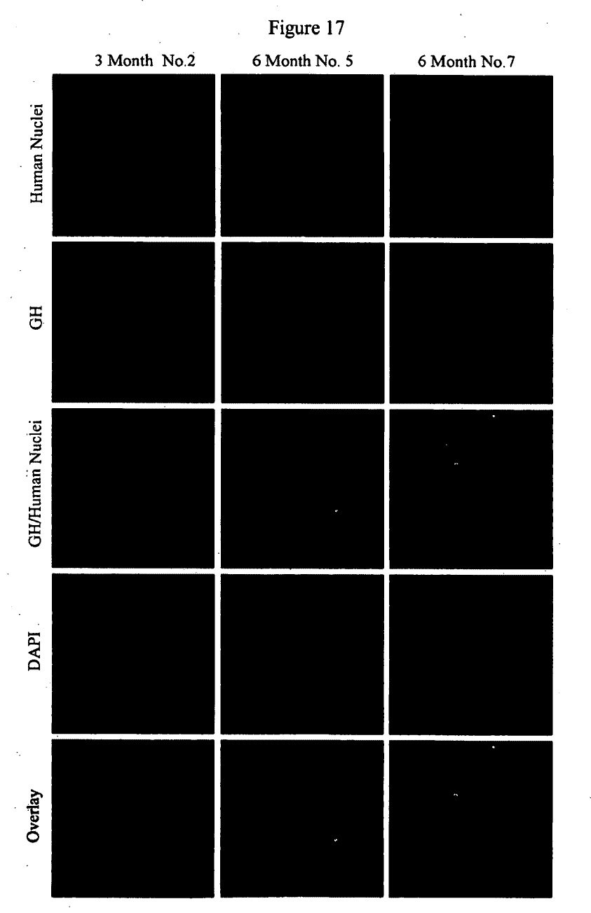

To study whether the tumor spheres have the ability to form new tumors in in vivo environment, tumor spheres (1×10⁴ cells per mouse) or monolayer non-sphere cells (1×10⁵ cells per mouse) were stereotactically implanted into the right hemisphere of NOD/SCID mice. Three months post the intracranial implantation, human-specific cells were identified in the brains of mice that received tumor spheres implantation. However, there was no human-specific cell found within the brains of mice that received monolayer non-sphere cells implantation. Six months post the intracranial implantation, larger areas of human-specific cell masses were identified in the brains of mice that received tumor spheres implantation compared with that of three months post the intracranial tumor spheres implantation. There were still no human-specific cells found within the brains of mice that received monolayer non-sphere cells: The identification of human-specific cells within the mice brains was performed by immunostaining with human-specific nuclei antibody against human cell nuclei (1:100, Chemicon, Temecula, Calif.). The antibody against human growth hormone (1:2000, Chemicon, Temecula, Calif.) was used to identify GH positive cells within the xenograft tumor mass. (See FIG. 17.)

### Example 20

### Tumor-Forming Ability was Confirmed by Serial in Vivo Transplantation

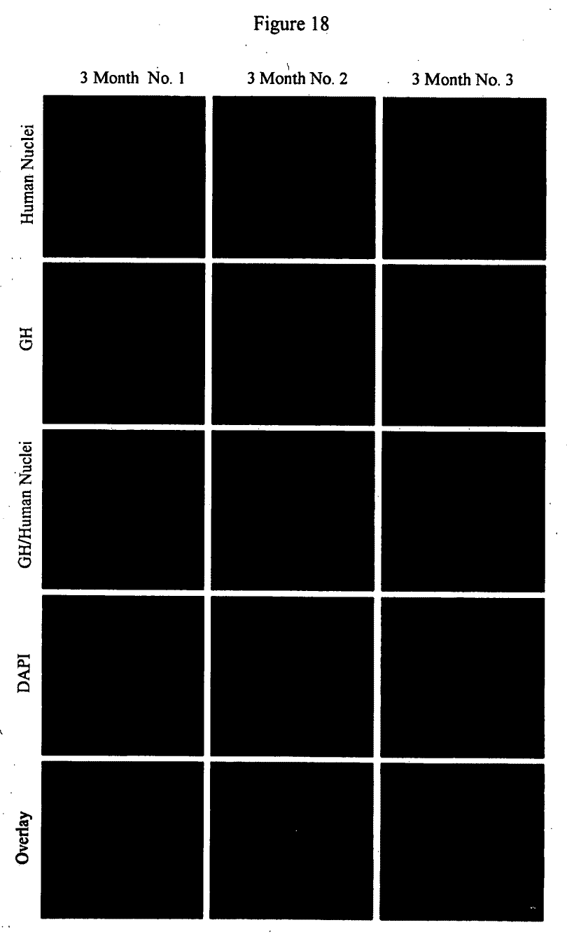

To investigate whether the ability to form new tumors was serially transplantable, the sphere-generated tumor masses within the NOD/SCID brains were harvested after 6 months of the intracranial implantation. The harvested tissues were primarily cultured as that of culturing human pituitary adenoma cells described herein. Sphere-growing cells were identifiable in the culture and the spheres can be propagated as free-floating spheres in stem cell culture medium. These spheres cells were re-transplanted into the brains of NOD/SCID mice (1×10⁴ cells per mouse). Three months post the transplantation, the mice were killed and the brain tissues were processed for human-specific cell identification. All three mice with the transplantation were found containing positive cell masses for human-specific nuclei antibody. Some cells within the masses were human growth hormone positive as well. (See FIG. 18.)

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. An isolated pituitary stem cell comprising cell markers selected from nestin, CD133 or both.

2. The isolated pituitary stem cell of claim 1, wherein the pituitary stem cell is a pituitary adenoma stem cell.

3. The pituitary adenoma stem cell of claim 2, wherein the pituitary adenoma is selected from the group consisting of prolactinoma, somatotrophic adenoma, adrenocorticotropic hormone (“ACTH”)-secreting adenoma, gonadrotophic adenoma, thyrotropic adenoma, null cell adenoma, and combinations thereof.

4. The isolated pituitary stem cell of claim 1, wherein the pituitary stem cell is a pituitary carcinoma stem cell.

5. The isolated pituitary stem cell of claim 1 obtained by:
providing pituitary tumor tissue;
washing the pituitary tumor tissue;
dissecting the pituitary tumor tissue;
digesting the pituitary tumor tissue;
triturating the pituitary tumor tissue to dissociate pituitary cells;
culturing the pituitary cells in a medium comprising EGF and bFGF; and
selecting the pituitary cells growing as spheres.

6. A method of obtaining a population of pituitary cells comprising:
providing a population of pituitary stem cells; and
culturing the population of pituitary stem cells in differentiation culture medium wherein the population of pituitary stem cells are induced to differentiate into pituitary cells.

7. The method of claim 6, wherein differentiation culture medium comprises DMEM/F12, glutamine, horse serum, and fetal bovine serum.

8. The method of claim 6, wherein the pituitary cells are pituitary adenoma cells.

9. The method of claim 8, wherein the pituitary adenoma cells are selected from the group consisting of prolactinoma, somatotrophic adenoma, adrenocorticotropic hormone (“ACTH”)-secreting adenoma, gonadrotophic adenoma, thyrotropic adenoma, null cell adenoma, and combinations thereof.

10. The method of claim 6, wherein the pituitary cells are pituitary carcinoma cells.

11. A method of producing a pituitary hormone, comprising:
providing a population of pituitary stem cells and/or pituitary cells obtained by differentiation of pituitary stem cells;
culturing the population in a culture medium; and
isolating the pituitary hormone from the culture medium or the intracellular contents of the pituitary stem cells and/or pituitary cells obtained by differentiation of pituitary stem cells.

12. The method of claim 11, wherein the pituitary hormone is selected from the group consisting of prolactin, growth hormone, adrenocorticotropic hormone, sexual hormone, and combinations thereof.

13. The method of claim 11, wherein the population is a population of cells selected from the group consisting of prolactinoma, somatotrophic adenoma, adrenocorticotropic hormone (“ACTH”)-secreting adenoma, gonadrotophic adenoma, thyrotropic adenoma, null cell adenoma, carcinoma and combinations thereof.

14. A method of identifying a drug to treat a pituitary disease condition or a pituitary-related disease condition, comprising:
providing a population of pituitary stem cells and/or pituitary cells obtained by differentiation of pituitary stem cells;
culturing the population in a culture medium;
adding a test compound to the culture medium; and
determining the effect of the test compound on the population,
wherein a test compound having a desired effect is identified as a drug capable of treating the pituitary disease condition or pituitary-related disease condition.

15. The method of claim 14, wherein the pituitary disease condition or pituitary-related disease condition is selected from the group consisting of pituitary adenoma, pituitary carcinoma, amenorrhea, galactorrhea, infertility, hypogonadism, gigantism, acromegaly, Cushing's disease, hyperthyroidism and combinations thereof.

16. The method of claim 14, wherein the pituitary stem cells are obtained from a pituitary adenoma or a pituitary carcinoma.

17. The method of claim 16, wherein the pituitary adenoma is selected from the group consisting of prolactinoma, somatotrophic adenoma, adrenocorticotropic hormone (“ACTH”)-secreting adenoma, gonadrotophic adenoma, thyrotropic adenoma, null cell adenoma, and combinations thereof.

18. The method of claim 14, wherein the population of pituitary stem cells is obtained by:
providing pituitary tumor tissue;
washing the pituitary tumor tissue;
dissecting the pituitary tumor tissue;
digesting the pituitary tumor tissue;
triturating the pituitary tumor tissue to dissociate pituitary cells;
culturing the pituitary cells in a medium comprising EGF and bFGF; and
selecting the pituitary cells growing as spheres.

19. The method of claim 14, wherein the population is a population of pituitary carcinoma stem cells and/or pituitary carcinoma cells obtained by differentiation of pituitary carcinoma stem cells and the drug is an anti-cancer drug.

20. A kit for producing a pituitary hormone using pituitary stem cells, comprising:
a population of pituitary stem cells and/or pituitary cells obtained by differentiation of pituitary stem cells;
instructions to use the population to produce the pituitary hormone comprising:
instructions to culture the population in a culture medium; and
instructions to isolate the pituitary hormone from the culture medium or the intracellular contents of the population.

21. The kit of claim 20, wherein the pituitary hormone is selected from the group consisting of prolactin, growth hormone, adrenocorticotropic hormone, sexual hormone, and combinations thereof.

22. The kit of claim 20, wherein the population is a population of cells selected from the group consisting of prolactinoma, somatotrophic adenoma, adrenocorticotropic hormone (“ACTH”)-secreting adenoma, gonadrotophic adenoma, thyrotropic adenoma, null cell adenoma, carcinoma and combinations thereof.


SEQUENCE LISTING (36 sequences)

SEQ ID NO: 1 - DNA, 21 bp, Artificial
  PRIMER
gccatcctggctgtcgagaag

SEQ ID NO: 2 - DNA, 21 bp, Artificial
  PRIMER
gagccggatggactcgaagcg

SEQ ID NO: 3 - DNA, 21 bp, Artificial
  PRIMER
acaatgcacatcgaaaggaga

SEQ ID NO: 4 - DNA, 21 bp, Artificial
  PRIMER
tcctggtatttctacagtcct

SEQ ID NO: 5 - DNA, 21 bp, Artificial
  PRIMER
atagagaaagaagaatgtcgt

SEQ ID NO: 6 - DNA, 21 bp, Artificial
  PRIMER
gtgagcacagccgggcactct

SEQ ID NO: 7 - DNA, 21 bp, Artificial
  PRIMER
agcttggccatatctgatatg

SEQ ID NO: 8 - DNA, 21 bp, Artificial
  PRIMER
gatgtagcggtccgcagcaat

SEQ ID NO: 9 - DNA, 27 bp, Artificial
  PRIMER
atgacacctatcaggagtttgaagaag

SEQ ID NO: 10 - DNA, 27 bp, Artificial
  PRIMER
gatgcggagcagctctaggttagattt

SEQ ID NO: 11 - DNA, 22 bp, Artificial
  PRIMER
gggttcattaccaaggccatca

SEQ ID NO: 12 - DNA, 21 bp, Artificial
  PRIMER
ttcaggatgaacctggctgac

SEQ ID NO: 13 - DNA, 21 bp, Artificial
  PRIMER
gagtcagcctttgggaggaac

SEQ ID NO: 14 - DNA, 21 bp, Artificial
  PRIMER
tggtggtggtggtgctgcgta

SEQ ID NO: 15 - DNA, 20 bp, Artificial
  PRIMER
acagctgctgatttcaagca

SEQ ID NO: 16 - DNA, 20 bp, Artificial
  PRIMER
acaaagctcctacttgctca

SEQ ID NO: 17 - DNA, 21 bp, Artificial
  PRIMER
ccctaagcagcgcagcaagac

SEQ ID NO: 18 - DNA, 21 bp, Artificial
  PRIMER
gatgagtggtcggttctggcc

SEQ ID NO: 19 - DNA, 21 bp, Artificial
  PRIMER
tccgctcctgatgtgcaggat

SEQ ID NO: 20 - DNA, 21 bp, Artificial
  PRIMER
ggaccttagtggagtgggata

SEQ ID NO: 21 - DNA, 18 bp, Artificial
  PRIMER
gaaggtgaaggtcggagt

SEQ ID NO: 22 - DNA, 20 bp, Artificial
  PRIMER
gaagatggtgatgggatttc

SEQ ID NO: 23 - DNA, 20 bp, Artificial
  PRIMER
cgtcttcaccaccatggaga

SEQ ID NO: 24 - DNA, 20 bp, Artificial
  PRIMER
cggccatcacgccacagttt

SEQ ID NO: 25 - DNA, 20 bp, Artificial
  PRIMER
agggaggaaagcagactgac

SEQ ID NO: 26 - DNA, 20 bp, Artificial
  PRIMER
ccagtcatttccacaccact

SEQ ID NO: 27 - DNA, 24 bp, Artificial
  PRIMER
tgtgatgtgggaaagcaggaggat

SEQ ID NO: 28 - DNA, 24 bp, Artificial
  PRIMER
acatgtgctggtctctggttacga

SEQ ID NO: 29 - DNA, 20 bp, Artificial
  PRIMER
cctgaagcagaagaggatca

SEQ ID NO: 30 - DNA, 20 bp, Artificial
  PRIMER
ccgcagcttacacatgttct

SEQ ID NO: 31 - DNA, 20 bp, Artificial
  PRIMER
aaccagcaaggaaaatccaa

SEQ ID NO: 32 - DNA, 20 bp, Artificial
  PRIMER
aggagcaggacgaagatgac

SEQ ID NO: 33 - DNA, 20 bp, Artificial
  PRIMER
accagctcgcagacctacat

SEQ ID NO: 34 - DNA, 20 bp, Artificial
  PRIMER
gtgggaggaagaggtaacca

SEQ ID NO: 35 - DNA, 26 bp, Artificial
  PRIMER
ggagaccagcaagtattgtccttttg

SEQ ID NO: 36 - DNA, 22 bp, Artificial
  PRIMER
cattgctgctgggcatcgtaag